(19) United States
(12) Patent Application Publication
(10) Pub. No.: US 2026/0053454 A1
(43) Pub. Date: Feb. 26, 2026

(54) MOTION-SENSING COUCH FOR RADIOTHERAPY SYSTEM

(71) Applicant: RefleXion Medical, Inc., Hayward, CA (US)

(72) Inventors: Christopher Eric BROWN, Morgan Hill, CA (US); Jackson Rand DUGONI, San Francisco, CA (US); George Andrew ZDASIUK, Portola Valley, CA (US)

(21) Appl. No.: 19/297,421

(22) Filed: Aug. 12, 2025

Related U.S. Application Data
(63) Continuation of application No. PCT/US2024/016723, filed on Feb. 21, 2024.
(60) Provisional application No. 63/486,859, filed on Feb. 24, 2023.

(51) Int. Cl. A61B 6/00; A61B 5/00; A61B 5/11; A61B 6/04; A61B 90/00

(52) U.S. Cl. CPC A61B 6/4447; A61B 5/11; A61B 5/725; A61B 6/0407; A61B 6/5276; A61B 90/06; A61B 2090/067

(57) ABSTRACT

Described herein are variations of a radiotherapy patient motion detection system. For example, a patient motion detection system may include a sensor coupled to a patient support platform for use in measuring patient motion during a radiotherapy treatment session. The motion sensor may transmit measurements to a controller for processing and subsequent use in timing delivery of radiation to a treatment region of interest within a patient. Also described herein are methods of use of a patient motion detection system.

### CROSS-REFERENCE TO RELATED APPLICATIONS

This application is a continuation of International Application No. PCT/US2024/016723, filed Feb. 21, 2024, which claims priority to U.S. Provisional Patent Application No. 63/486,859 filed Feb. 24, 2023, the disclosure of which is hereby incorporated by reference in its entirety.

### TECHNICAL FIELD

Described herein are systems and methods for monitoring patient motion and control during radiation therapy. The systems and methods may be used for emission-guided high-energy photon delivery.

### BACKGROUND

Radiotherapy, or radiation therapy, uses high-energy photons to treat a variety of diseases. For instance, radiotherapy is commonly applied to cancerous tumors. To receive treatment, a patient may be placed on a patient platform that moves in and out of a gantry which moves around the patient and emits radiation beams to a treatment region of interest within the patient. Accurately tracking the treatment region (e.g., tumor position) is a critically important factor when maximizing radiation dose to the treatment region and limiting radiation exposure to a patient's normal tissue. Patient motion, including large motions (e.g., coughing, rolling over, etc.), peristaltic motion and respiration, can cause movement of the treatment region and complicate this task. To overcome patient motion difficulties, some radiotherapy systems use motion detection systems to facilitate delivery of radiation to the treatment region. For example, a respiratory gating system tracks a patient's respiratory cycle with imaging and/or markers and delivers radiation only when the treatment region is within a treatment field. However, traditional patient motion detection systems are typically separate from companion radiotherapy treatment systems and add complexity to the workflow as they require a sensor to be attached to a patient or a patient platform prior to each treatment session. Other systems may require the projection of a light beam or other form of radiation onto the patient requiring the removal of clothing or other means of patient preparation. Further, patient motion data collected from such separate systems must be subsequently coordinated and/or interfaced with companion radiotherapy systems to deliver modified therapy considering patient motion. Accordingly, there is a need for a new and improved radiotherapy patient motion detection systems and methods of using the information obtained thereby.

### BRIEF SUMMARY

In some variations, a system for detecting patient motion during a radiotherapy treatment session may include a gantry with a radiation source, a patient platform with a patient support surface, where the patient platform may translate laterally through the gantry, and a sensor coupled to the patient support surface, where the patient platform sensor may collect data of a patient on the patient support surface. In some variations, the sensor may be communicably coupled to a controller with a memory which may store the motion data. In some variations, the sensor may include a memory which may store the motion data. In some variations, the motion data may be stored in the memory as a CSV file. In some variations, the motion data may include a magnitude of one or more of a slope, an elevation, a depression, a force, or a strain of the patient platform. In some variations, the motion data may be used to modify a radiation dose delivered to the patient. For example, the motion data may be used to modify the timing when a radiation beam is emitted and/or the shape, size and/or location of the radiation beam. In some variations, the patient platform sensor may be a motion sensor. In some variations, the patient platform sensor may collect the motion data at a rate of between about 1 Hz and about 100 Hz. In some variations, the patient platform sensor may collect the motion data at a rate of at least 50 Hz.

In some variations, the patient platform sensor may be an inclinometer. In some variations, the inclinometer may measure an angle of one or more of a slope, an elevation, or a depression of the patient platform. In some variations, a resolution of the inclinometer may be between about 1 μradian and about 5 μradians.

In some variations, a radiotherapy system for detecting patient motion may include a platform which may support a patient during a radiotherapy treatment session, the patient platform having a patient support surface and a sensor coupled to the patient support surface, where the sensor may collect data on the patient motion data (and optionally, couch motion and/or location data) during the radiotherapy treatment session. In some variations, the sensor may be communicably coupled to a controller with a memory which may store the data. In some variations, the data may be used to modify a radiation dose delivered to the patient.

In some variations, the system may also include a processor (e.g., a processor of the controller) which may apply a filter to the data. In some variations, the filter may be a first-order filter. In some variations, the processor may be configured to denoise and resample the data.

In some variations, the sensor may be an inclinometer. In some variations, the inclinometer may measure an angle of one or more of a slope, an elevation, or a depression of the patient platform. In some variations, a resolution of the inclinometer may be about 3.5 μradians.

In some variations, a method for detecting patient motion during a radiotherapy session may include collecting patient motion data from a patient on a patient platform, where the patient platform may include a patient support surface and a sensor coupled to the patient support surface, and where the patient motion data may be collected by the sensor, storing the patient motion data, and optionally modifying a radiation therapy delivered to the patient based at least in part on the patient motion data. In some variations, modifying the radiation therapy may include respiratory gating. In some variations, modifying the radiation therapy may include delaying or completely stopping a delivery of a radiotherapy beam during a large motion made by the patient. In some variations, the sensor may detect the data on the patient motion at a rate of about 80 Hz or more, e.g., 100 Hz or more, 125 Hz or more. In some variations, the patient motion may include one or more of a breath, a cough, a torso adjustment, a limb adjustment, and a head adjustment.

In some variations, modifying the radiation therapy may include delivering a radiotherapy beam to the patient during a specific breathing phase of the patient. In some variations, a specific breathing phase of the patient comprises a breath-hold.

In some variations, the patient motion may include a change in a center of mass of the patient with respect to the patient platform. In some variations, the change in the center of mass of the patient may cause a change in a strain field of the patient platform.

### BRIEF DESCRIPTION OF THE DRAWINGS

FIG. 1 depicts a cross-sectional view of a variation of a radiotherapy patient motion detection system.

FIG. 2 depicts a variation of a radiotherapy patient motion detection system including an inclinometer, an optical encoder, a resolver, a strain gauge, and a strain gauge load cell.

FIG. 3 depicts a variation of a controller for use in a patient motion detection system.

FIG. 4 depicts a flowchart representation of a variation of method for detecting motion on a couch during a radiation delivery session, without a patient or with a patient (e.g., to detect patient motion during radiotherapy).

FIGS. 5A-5D depict graphical representations of four unfiltered outputs of a patient motion detection system.

FIGS. 6A-6D depict graphical representations of four filtered outputs of a patient motion detection system.

FIGS. 7A and 7B depict graphical representations of unfiltered and filtered measurements corresponding to patient movement on a patient platform.

FIGS. 8A and 8B depict graphical representations of unfiltered and filtered measurements corresponding to patient respiration on a patient platform.

FIG. 9 depicts a variation of a radiotherapy treatment system and integrated patient motion detection system.

FIGS. 10A and 10B depict graphical representations of two different filtering methods applied to outputs of a patient motion detection system.

FIGS. 11A and 11B depict graphical representations of two different filtering methods applied to outputs of a patient motion detection system.

FIGS. 12A and 12B depict graphical representations of filtered outputs of a patient motion detection system.

FIGS. 13A and 13B depict graphical representations of filtered outputs of a patient motion detection system.

### DETAILED DESCRIPTION

Nonlimiting examples of various aspects and variations of the invention are described herein and illustrated in the accompanying drawings.

Many medical procedures benefit from minimizing and/or monitoring patient motion to treat or diagnose patients safely and accurately. Often, patient support platforms (e.g., beds, chairs, couches) are used to stabilize a patient during a procedure or diagnostic session. For example, surgical procedures typically occur as a patient is lying or sitting on a support platform to minimize patient movement and thereby increase the safety of the procedure. As another example, diagnostic and interventional medical imaging sessions (using, e.g., X-ray imaging, PET, MRI, etc.) typically occur as a patient is lying or sitting on a support platform to minimize patient movement such that a resultant image accurately depicts an anatomical region of interest.

Radiotherapy is an interventional procedure which delivers radiation beams to a treatment region (e.g., legion, tumor). In some variations, radiotherapy methods may use real-time imaging techniques to guide radiation beam delivery. Monitoring patient motion during a radiotherapy session is important to minimize delivery of radiation to healthy tissue, which may encroach on a target treatment zone as a result of even small patient motion (e.g., respiration, peristalsis). Patient motion may involve variations in a patient's position (e.g., center of mass), which may be monitored according to systems and methods described herein to reduce delivery of radiation to a patient's healthy tissue during a radiotherapy treatment session. Optionally, the systems and methods described herein allow for modification of a radiation beam delivered to the target treatment zone (e.g., changing one or more beam properties, delaying delivery of a beam to the treatment zone) based on variations of the patient's position. Although the patient motion detection systems and methods described herein may be described with respect to radiotherapy systems, it should be understood that such systems may additionally or alternatively be configured for use with other medical treatment and/or diagnostic systems, such as medical imaging systems (e.g., X-ray imaging, PET, MRI, etc.) or surgical systems.

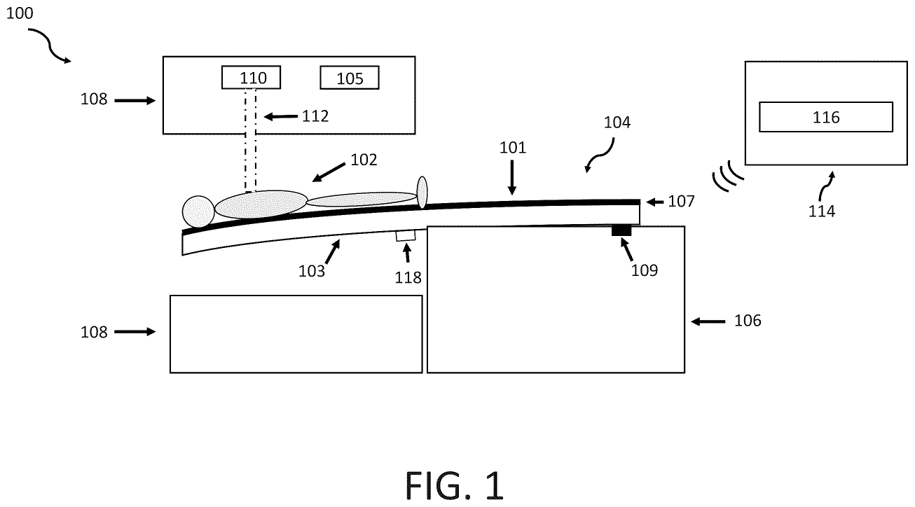

Generally, to perform a radiotherapy procedure, a patient is loaded onto a patient platform (e.g., couch) that is moveable with respect to a gantry. The gantry may include a bore (e.g., central opening, cavity) and one or more beam delivery systems that may rotate about the patient platform and provide one or more radiation treatment beams from a plurality of angles. FIG. 1 shows a cross-sectional view of one variation of a radiotherapy treatment system (i.e., radiotherapy delivery system) and integrated patient motion detection system. Radiotherapy treatment system 100 may include a patient platform 104 which may be supported by base 106. During a session (e.g., treatment or imaging session), a patient 102 may be positioned on the patient platform 104. The patient platform may be laterally and rotationally movable within a bore of the gantry (or a patient area) and may include a patient support surface 101 (i.e., top side) on which the patient may lie and a bottom surface 103 (i.e., underside). The patient support surface may optionally include cushion 107 to help secure the patient in place on the support surface and aid in patient comfort. Additionally, the patient platform may include one or more sensors 118 to capture data, such as patient motion data and/or couch position and/or couch motion data, which may be used to facilitate monitoring and/or modifying the treatment delivered to the patient. The gantry may include radiation source 110 to emit radiation beam 112 to treat a region of interest within the patient. The gantry may be a circular gantry or a C-arm gantry, and/or may comprise one or more robotic arms configured to position the radiation source 110 relative to the patient area (e.g., bore). The radiation source 110 may be a therapeutic radiation source that is configured to emit high-energy radiation, e.g., a linear accelerator that emits radiation beams between about 2 MV to about 20 MV, such as a radiation beam of about 6 MV. In some variations, such as when proton beams are used for delivering therapeutic radiation, radiation source 110 may emit beams between about 20 MV and about 300 MV. Further, controller 114 may direct delivery of radiation via communication module 116 and based at least in part on patient movement during a treatment session. For example, communication module 116 may transmit control signals to communication interface 105 on the gantry to guide a radiotherapy session. The control signals may be transmitted wirelessly and/or using an electrical cable. Monitoring patient motion to safely deliver radiotherapy to a treatment region of the patient may include accounting for any sag, deviation, and/or deflection of the patient platform (within an acceptable tolerance), which may provide information about the patient's position and/or motion. As described in further detail below, accounting for patient motion may include accounting for mechanical and/or rotational changes of the patient platform resulting from changes in a position (e.g., center of mass) of the patient.

As generally described herein, a patient motion detection system for use in monitoring and/or modifying a radiotherapy treatment may include a patient platform having a patient support surface and an embedded sensor to detect patient motion during a radiotherapy treatment session. The sensor may be embedded in a portion of the patient platform, e.g., attached or coupled to the patient support surface. Patient motion on the patient platform may correspond to mechanical and/or rotational changes of the platform because patient motion (e.g., coughing, sitting up, respiration, peristalsis, etc.) may involve changes in the patient's center of mass with respect to the platform coordinates. For example, a change in the patient's center of mass may cause a detectable change in the strain field of the patient platform due to the shifting force that the patient applies to the platform. As another example, a change in a patient's center of mass may cause a detectable change in a slope of the patient platform due to the shifting force applied by the patient. Additionally, a change in a patient's center of mass may cause a detectable change in a resisting force of the platform if the platform is cantilevered at a pivot point. Alternatively, if the platform is supported by independent actuators, the forces applied to the platform by the actuators to keep the patient in position may also vary with changes in the patient's center of mass. Accordingly, measuring properties of the patient platform (e.g., strain within the platform, pitch of the platform) at a suitable frequency may help guide the delivery of radiation to a target treatment zone within the patient. For example, if the patient motion detection system identifies a patient motion, the system or a medical professional may modify a radiation beam delivered to the patient to minimize delivery of radiation to the patient's healthy tissue, which may have moved into the target treatment zone due to the patient motion. Thus, the platform-embedded (e.g., platform-mounted) sensor may, for example, measure an angle of slope, elevation, strain, resisting force or depression of the patient platform, which may indicate a deviation or deflection of the patient in the treatment beam plane. The sensor may transmit mechanical and/or rotational measurements of the patient platform to a controller which may process the measurements and use them to adjust and/or determine timing of delivery of radiation treatment to the patient.

The patient motion detection systems described herein may thus be useful in improving numerous typical radiotherapy treatments. For example, typical radiotherapy patient motion detection systems are separate from the patient platform and must be attached directly to the patient or to the patient platform prior to each treatment session. Another example of a separate motion detection system may include cameras and light sources mounted on the ceiling, wall, and/or floor directed toward the patient to determine the patient's position and motion. However, data collected by such separate motion detection systems must be coordinated (i.e., calibrated and synchronized) with the radiotherapy delivery system. In contrast, the patient motion detection systems described herein utilize on-board patient platform sensors that are integrated with the radiotherapy delivery system, reducing the complexity of radiotherapy treatment workflow. An on-board patient platform sensor may also be on the same coordinate system as the radiotherapy system, so that the readings from the on-board sensor are registered with respect to the radiotherapy system coordinate system. A simplified workflow may allow an increased number of radiotherapy treatment sessions to occur over time (e.g., over a day), thereby supporting the treatment of more patients. Moreover, the systems described herein may utilize sensors that are sensitive enough to detect even small patient motions (e.g., motion due to breathing, peristalsis), which may increase the safety of radiotherapy, for example, by making more accurate and/or frequent adjustments to reduce delivery of radiation to healthy tissue.

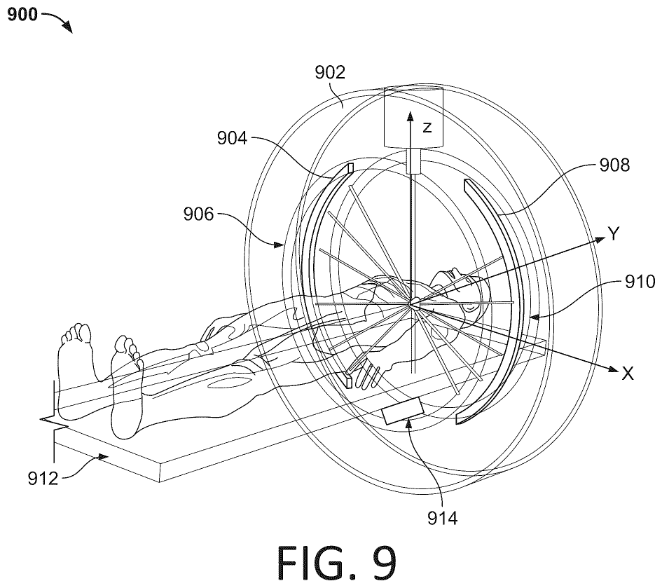

Another variation of a radiotherapy treatment system with integrated patient motion detection system is shown in FIG. 9. System 900 includes gantry 902, which may also include one or more mounted PET detectors. For example, gantry 902 may include a first array 904 of PET detectors mounted along a first length 906 of the circumference (i.e., inner circumference) of the gantry and a second array 908 of PET detectors mounted along a second length 910 of the circumference (i.e., inner circumference) of the gantry. Further, system 900 may include patient platform 912 with mounted sensor 914, where the patient platform is movable within the gantry.

Throughout this application, the term “integrated” is used to describe a patient motion detection system which is completely (mechanically and electrically) consolidated into a radiotherapy treatment system such that they function as a single radiotherapy treatment system. Such a unified system would simplify (and in some cases, eliminate) the synchronization and/or coordinate registration procedures for connecting the patient motion detection system to the radiotherapy treatment system. For example, a patient motion detection system may not be integrated with a radiotherapy treatment system if it requires one or more of: synchronization of the patient motion detection system to the radiotherapy treatment system, or temporary attachment of a patient motion detection system component (e.g., a sensor) to a patient or radiotherapy treatment system component (e.g., patient platform, gantry) prior to or during a treatment session.

Various aspects of example variations of radiotherapy patient motion detection systems and methods of use thereof are described in further detail below.

### Patient Motion Detection System

As shown in FIG. 1, a radiotherapy patient motion detection system, which is integrated with radiotherapy treatment system 100, may include sensor 118 fixed to patient platform 104 and controller 114 that is in communication with the patient motion detection system (including the sensor) and other components of the radiotherapy system to control its operation. In one variation, the controller 114 may optionally be programmed and/or otherwise configured to modify the radiotherapy treatment using patient motion data detected by the sensor. The sensor may be a digital or analog motion sensor configured to measure motion and/or rotational properties of the patient platform such as an inclinometer, an encoder, a resolver, a synchro, a transducer, or any other type of rotary position or motion feedback sensor. Additionally, or alternatively, the sensor may be a digital or analog mechanical sensor configured to measure one or more mechanical properties of the patient platform and be configured as a strain gauge, stress gauge, alignment meter, thermal sensor, pressure probe, piezoelectric sensor and/or the like. In some variations, the sensor may be a microelectromechanical sensor. In variations where the sensor includes an analog front end, the sensor may comprise circuitry to convert analog measurements to digital measurements suitable for transmission to the controller. Sensor measurements may be discrete values, continuous values, or may have the option of being discrete or continuous values. Further, the sensor may have a high resolution to measure a range of patient motion with respect to the patient platform including, but not limited to, respiration, coughing, sneezing, and moving one or more body part. In some variations, the sensor may include more than one sensor and/or a combination of sensors to measure various properties of the patient platform, such as mechanical properties of the patient platform including, but not limited to, deflection, deformation, strain, and/or stress sustained by the patient platform in response to changes in a patient's center of mass or a redistribution of the patient's weight profile on the table. In some variations, the sensor may be configured to measure a property of the patient platform and/or transmit measurements of the patient platform to the controller at a predetermined rate. Typically, the sensor may include a transmitter to convert sensor measurements to electrical signal outputs and transmit the outputs to a controller. In some variations, the sensor may further include a memory to store captured patient motion data.

In contrast to traditional radiotherapy patient motion detection systems, which include removable sensors temporarily attached to a patient or patient platform prior to treatment, the patient motion detection systems described herein may include a sensor that is, or a plurality of sensors that are, fixed to the patient platform (e.g., sensor 118 in FIG. 1) and thus integrated with a radiotherapy treatment system. For example, the sensor may be mounted to an underside of a support surface of the patient platform, mounted adjacent to the patient platform such that it is in direct contact with the support surface of the patient platform, embedded within the patient support surface, embedded within a substrate coupled to the patient support surface, or fixed by any other suitable fixation mechanism to the patient platform. The integrated sensor may be fixed to the platform such that it is used for multiple treatment sessions and/or for multiple patients, with the intention that it is removed and replaced primarily for calibration and/or repair purposes. In some variations, a mechanism of attaching a sensor to a patient platform may include nailing, gluing, fusing, sewing, screwing, embedding, or using magnetic forces to fix the sensor to the platform. In other variations the sensor is connected to the platform via friction or other mechanical fit. The sensor may be fixed to any part of the patient platform, including the underside, the top side, a sidewall, within a platform substrate, or within a platform cushion. For example, a sensor may be located between a patient's center of mass and a fixed pivot point on the platform. In variations where the patient platform may be supported at multiple points, a sensor (e.g., a load cell) may be placed between each of multiple supporting members and the patient platform at each of the multiple support points. In some variations, the sensor may be attached to a portion of the patient platform that is not movable into the radiation treatment plane, which may help limit radiation damage to the sensor. For example, the sensor may be attached to the patient platform at the end furthest from the bore and/or closer to the base that is supporting the patient platform.

In general, a controller for use with the patient motion detection systems described herein, such as controller 114 in FIG. 1, may receive, store, process, and use patient motion data detected by the sensor to modify a radiotherapy treatment delivered to a patient. For example, the controller may receive, store, process, and use sensor data corresponding to a large patient motion (e.g., movement of a limb, a cough, etc.) to delay a delivery of a radiation beam to a treatment region within a patient until the sensor data corresponds to no patient movement. As another example, the controller may receive, store, process, and use sensor data corresponding to a patient's breathing motion (e.g., inhale, exhale, breath-hold) to delay a delivery of a radiation beam to a treatment region within a patient until the sensor data corresponds to a breath-hold. Additionally, or alternatively, the controller may deliver the radiation beam along a different path to account for patient movement that results in movement of the treatment region. Accordingly, the controller may include a memory to store sensor data, one or more processors to perform analysis on the sensor data, and/or a communication module (e.g., wireless communication module) configured to communicate the sensor data to the radiotherapy treatment system. In some variations, the controller may be integrated with the radiotherapy treatment system and may automatically modify the treatment based at least in part on the patient motion data. Additionally, or alternatively, a medical professional may have access to the patient motion data via the controller and may assess the data to inform decisions regarding treatment modification. For example, the controller may include a display configured to show a user interface though which a medical professional may input a decision regarding radiation delivery (e.g., deliver radiation, delay radiation, change radiation delivery profile, stop the treatment, resume treatment etc.). In some variations, the user interface may also allow a medical professional to query a patient motion detection system sensor for a measurement related to patient motion at any time.

Additional aspects of patient motion detection systems are described in further detail below.

### Sensor

As described above, a patient motion detection system of a radiotherapy system may include an embedded or integrated sensor. For example, the embedded or integrated sensor may be fixed to a patient platform. The patient motion detection system may include one or more sensors and/or a combination of various sensors. The one or more sensors may be a digital, analog sensors, or a combination of digital and analog sensors. Nonlimiting examples of sensors and sensor combinations for use with this technology are described in further detail below.

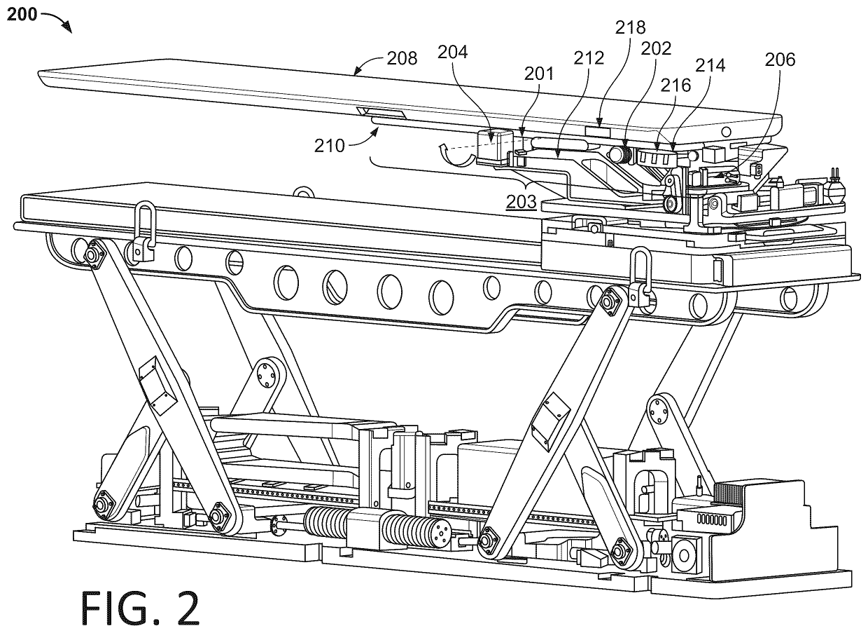

FIG. 2 depicts one variation of a motion sensing patient platform 200 that may be used with a radiotherapy system. The patient platform 200 may have patient support surface 208 and bottom surface 210 to which one or more sensor(s) (e.g., inclinometer 202, optical encoder 204, integrated rotary system and resolver 206, strain gauge load cell 214, strain gauge 218) may be fixedly or removably attached. Any one of inclinometer 202, optical encoder 204, resolver 206, strain gauge load cell 214, and strain gauge 218 may be used (alone or in combination) to help detect patient motion via, e.g., measurements of rotational or mechanical responses of patient platform 200 to the patient motion. While FIG. 2 depicts multiple sensors integrated with the patient platform 200, it should be understood that in some variations, a patient platform may have only one of these types of sensors (e.g., solely an inclinometer, solely a strain gauge, solely an optical encoder, etc.) or two of these types of sensors (e.g., optical encoder, inclinometer) or three of these sensors (e.g., optical encoder, inclinometer, resolver). Further, one or more of the sensors may be used in any combination in the patient motion detection system to increase the amount of patient motion data captured by the system. This may in turn increase the breadth and accuracy of patient motion data used to inform radiotherapy treatment modifications by the system. While FIG. 2 shows that the one or more sensors may be attached to the bottom surface of distal base portion 203 of the patient platform 200 (indicated with a bracket), it should be understood that the one or more sensors may be attached to any location on the patient platform 200 (e.g., bottom surface 210, support surface 208, embedded within a substrate or cushion of the platform) to detect rotational and/or mechanical platform data associated with different platform locations. In some variations, the one or more sensors may be affixed to the distal base portion 203, which may help reduce and/or eliminate the radiation exposure of the one or more affixed sensors due to radiation emitted by the therapeutic radiation source.

Although FIG. 2 depicts use of inclinometer 202, optical encoder 204, resolver 206, and/or strain gauge load cell 214 to measure patient platform properties corresponding to patient motion, it should be appreciated that any suitable type of sensor may be used to detect changes in properties of the patient platform corresponding to patient movement on the platform. It should be understood that a patient motion detection system may use just one sensor or may use two or more sensors to measure the properties of the patient platform that correlate with patient motion.

### Inclinometer

An inclinometer may detect patient motion via measurements of one or more of a slope, an elevation, a depression, or combinations thereof, of the patient platform. The inclinometer may be configured to measure such patient platform angles at a rate of between about 1 Hz and about 500 Hz, such as between about 10 Hz and about 250 Hz, between about 50 Hz and about 100 Hz, or between about 70 Hz and about 90 Hz. For example, the inclinometer may measure one or more of a slope, an elevation, or a depression of the patient platform at a rate of about 80 Hz. In some variations, an inclinometer may include a memory to store angular measurements of the patient platform.

As shown in FIG. 2, inclinometer 202 may be mounted to bottom surface 210 of the patient platform via any suitable attachment mechanism including screwing, nailing, or fusing at least a portion of the sensor to the bottom surface. Additionally, inclinometer 202 may include a horizontal and/or vertical mount. For example, inclinometer 202 may include a base with one or more portions extending horizontally and/or vertically. As shown in FIG. 2, inclinometer 202 may be attached adjacent to patient platform pitch drive system 212, which may be configured to raise and/or lower the patient platform. Further, inclinometer 202 may include an antenna (e.g., transmitter) to send detected patient platform inclination data to a companion controller.

Advantages of the inclinometer for use in the patient motion detection system may include its sensitivity (i.e., large input range and high resolution) and low weight. The inclinometer may have an angular input range of about 0° to about ±100°, such as an input range of about ±1° to about ±98°, an input range of about +2° to about ±94°, or an input range of about ±3° to about ±90°. The resolution of the inclinometer may be between about 1 μradian and about 10 μradians, such as between about 1.5 μradians and about 8 μradians, between about 2 μradians and about 7 μradians, between about 2.5 μradians and about 6 μradians, between about 3 μradians and about 5 μradians, or between about 3.5 μradians and about 4 μradians. For example, the inclinometer may have a resolution of about 3.5 μradians, which may be advantageous for perceiving small-scale patient motion (e.g., normal respiration) with respect to the patient platform. The weight of the inclinometer may be on the order of ounces, such as between about 0.5 oz and about 10 oz, between about 1.5 oz and about 8.5 oz, between about 2.5 oz and about 6.5 oz, or between about 3.5 oz and about 4.5 oz. For example, a lightweight inclinometer of about 4.2 oz may be mounted to a patient platform without interfering with functions of the platform, including its drive systems (e.g., pitch drive system 212 in FIG. 2). The inclinometer 202 may be mounted to the distal portion of the patient platform (e.g., near the base of the platform in the region bracketed by 203), which may help limit its exposure to radiation emitted by the therapeutic radiation source.

The angular data measured by the inclinometer may be transmitted to a companion controller, which may process the data to analyze patient motion during a radiotherapy session prior to modifying a radiotherapy delivered to a patient based at least in part on the angular data. For example, the inclinometer may include a 4-20 mA transmitter that converts angular patient platform measurements into equivalent 4-20 mA current loop output signal sent to a companion controller. The signal may be transmitted via an electrical cable or wirelessly. The controller may include one or more processors to process (e.g., filter) the inclinometer data, as described in further detail below.

### Optical Encoder

Also shown in FIG. 2 is optical encoder 204, which may detect rotational position information about patient platform 200 as an optical pulse signal. Generally, an optical encoder (i.e., encoder) includes a disc (i.e., code wheel) with a fixed slit attached to a rotating shaft (e.g., motor). An optical pulse may be generated by the encoder depending on whether light emitted from a fixed light emitting element (e.g., LED) passes through the fixed slit of the disc. A photosensor (e.g., photodiode, phototransistor) may detect the optical pulse, convert it into an electrical signal, and output the signal. The resolution of an optical encoder may increase as the number of slits in its disc increase and is a sensor that is useful for applications requiring high precision, accuracy, and/or resolution.

In some variations, optical encoder 204 may be a pitch optical encoder that detects rotation of patient platform 200 about the side-to-side axis 201. Optical encoder 204 may be configured to measure rotation of patient platform 200 at a rate of between about 1 Hz and about 500 Hz, such as between about 10 Hz and about 250 Hz, between about 50 Hz and about 100 Hz, or between about 70 Hz and about 90 Hz. Typically, optical encoder 204 may have quadrature detection, which may significantly increase its resolution. In some variations, optical encoder 204 may be a transmissive encoder as described above, which may have high signal accuracy. Alternatively, optical encoder 204 may be a reflective encoder in which the photosensor detects whether the light emitted from the light emitting element is reflected by the disc or not. In such alternative variations, optical encoder 204 may be lighter weight than traditional transmissive encoders. As described above in reference to inclinometer 202, optical encoder 204 may be fixedly attached to a surface of patient platform 200, including patient support surface 208. Optical encoder 204 may include a base, such as a mounting bracket, to attach it to patient platform 200 via any suitable attachment mechanism, e.g., screws, nails, glue or adhesive, friction fit, or the like. As described with respect to inclinometer 202, in general, the rotational data measured by optical encoder 204 may be converted into an electrical signal output (i.e., current, voltage) and transmitted to a companion controller via a transmitter.

### Resolver

Also shown in FIG. 2 is an integrated rotary system and resolver 206. The rotary system may drive an actuator which sets the angle of rotation of patient platform 200 in response to patient motion. In some variations, the rotary system may be a motor coupled to the resolver. The resolver in this integrated part may include a stator with windings. Inductive coupling between the windings may vary according to the angular position of the rotor. The resolver may be energized (e.g., with AC signal), and the resulting output from the windings may be converted to an electrical signal proportional to angle. The resolver may be any suitable resolver type including a brushless resolver, a slab resolver, a pancake resolver, a receiver resolver, or a differential resolver. The resolver may be configured to measure the angle of rotation of patient platform 200 at a rate of between about 1 Hz and about 500 Hz, such as between about 10 Hz and about 250 Hz, between about 50 Hz and about 100 Hz, or between about 70 Hz and about 90 Hz.

The integrated rotary system and resolver 206 may be attached to a surface of patient platform 200, such as bottom surface 210, and adjacent to a pitch actuator. An attached rotary system and resolver may include a base, such as a mounting bracket, to attach to patient platform 200 via screws, nails, or any other suitable attachment mechanism. Additionally, resolver 206 may include a transmitter to convert the measured angular data into an electrical signal output and transmit the data to a companion controller.

### Strain Gauge and Load Cell

As noted above, the patient motion detection systems described herein may include one or more mechanical sensors such as a strain gauge, a load cell, a piezoresistive force sensor, or a transducer that converts mechanical deformation into an electrical output.

A strain gauge may detect strain by undergoing a change in resistance when strain is applied to it. As shown in FIG. 2, strain gauge 218 may be embedded in patient support surface 208 to measure platform strain in response to variations of a patient's position (e.g., center of mass) and/or motion. Additionally, or alternatively, one or more strain gauge may be embedded within a cushion (not shown) atop support surface 208, or mounted on bottom surface 210 of patient platform 200. For example, strain gauge 218 may be used to measure strain of the substrate of the patient platform and/or pressure of a patient on the cushion on the patient platform. In some variations, strain gauge 218 may include a plurality of strain gauges embedded with a substrate or cushion (not shown) of patient platform 200. In some variations, strain gauge 218 may be a linear strain gauge.

Additionally, changes in the patient's center of mass may affect forces that the platform pitch actuator (e.g., pitch actuator 109 in FIG. 1) applies to the patient platform to maintain its position. FIG. 2 shows load cell 214, which typically includes a plurality of strain gauges 216, to detect an amount of force applied to it, and may be affixed to a patient platform between a pitch actuator and the platform to measure forces applied by the pitch actuator to the patient platform in response to variations in a patient's center of mass. Load cell 214 may be fixedly attached to a surface of patient platform 200, including bottom surface 210 and support surface 208. Load cell 214 may include a base, such as a mounting bracket, to attach to patient platform 200 via screws, nails, or any other suitable attachment mechanism. In some variations, the load cell may be affixed to both the patient platform and a platform pitch actuator.

Thus, both resistive forces of the patient platform and/or applied forces to the patient platform may be detected using one or more of these sensors. In some variations, load cell 214 and strain gauge 218 may be communicably coupled to and transmit and receive respective mechanical force measurements. Strain gauge 218 and/or load cell 214 may include a transmitter to send converted electrical strain and/or force data to a controller of the patient motion detection system.

Additionally, either one of strain gauge 218 and load cell 214 may be configured to measure strain or resistive force of patient platform 200 at a rate of between about 1 Hz and about 500 Hz, such as between about 10 Hz and about 250 Hz, between about 50 Hz and about 100 Hz, or between about 70 Hz and about 90 Hz.

### Controller

Generally, the systems described herein may comprise a controller configured to perform one or more steps of a radiotherapy procedure. The controller may be coupled (e.g., via an electrical cable or a wireless communication module) to one or more patient platform sensors (e.g., inclinometer(s) and the like) and the radiotherapy gantry. For example, a controller may be configured to initiate performance of one or more steps of a radiotherapy procedure including delivering a radiation beam based at least in part on patient movement captured by the sensors during a radiotherapy session. The controller may be disposed in the patient platform, a user console, or may be a separate device communicably coupled to one or more sensors and a computing device. In some variations, the controller may be configured to receive sensor output data (e.g., electrical current) simultaneously from one or more sensors. Additionally, the controller may be configured to store and process sensor data from one or more sensors in the patient motion detection system.

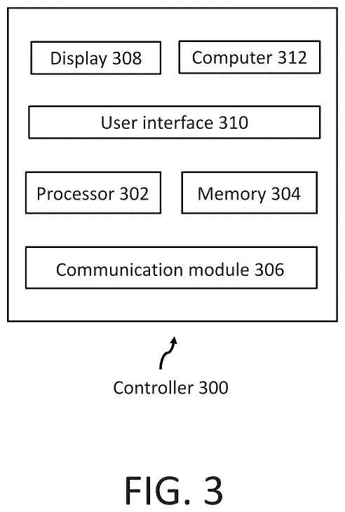

FIG. 3 shows an illustrative controller for use with patient motion detection systems and/or radiotherapy systems described herein. Controller 300 may include processor 302, memory 304, and communication module 306. In some variations, controller 300 may include one or more processors in communication with one or more machine-readable memories. Processor 302 may incorporate data received from memory 304 and patient input to control the treatment system. For example, memory 304 may store patient motion data detected by a sensor and received at communication module 306 for use in one or more processor modules, processes, and/or functions. Memory 304 may further store instructions to enable the processor to execute modules, processes and/or functions associated with the radiotherapy treatment system. Controller 300 may send a control signal to one or more parts of the treatment system via communication module 306 to guide a radiotherapy treatment session for a patient. For example, the controller may delay or modify delivery of a radiation beam to a treatment region of interest on a patient based at least in part on patient motion data transmitted to the controller by a sensor embedded in a patient platform. Further, controller 300 may include display 308 configured to support user interface 310. The user interface may allow a user, such as a physician overseeing a radiotherapy treatment, to view, modify, or otherwise interact with radiotherapy treatment data, variables, and/or controls. For example, display 308 may show patient motion data during a radiotherapy session, and user interface 310 may allow a physician to choose when to deliver a next radiation beam to a treatment region of interest within the patient based at least in part on the patient motion data.

The controller may be implemented with general purpose or special purpose computing systems or configurations. Various exemplary computing systems, environments, and/or configurations that may be suitable for use with the systems and devices disclosed herein may include, but are not limited to, software or other components within or embodied on a user console, servers or server computing devices such as routing/connectivity components, multiprocessor systems, microprocessor-based systems, distributed computing networks, personal computing devices, network appliances, portable (e.g., hand-held) devices, and the like.

The processor may be any suitable processing device configured to run and/or execute a set of instructions or code and may include one or more data processors, image processors, graphics processing units, physics processing units, digital signal processors, and/or central processing units. The processor may be, for example, a general-purpose processor, Field Programmable Gate Array (FPGA), an Application Specific Integrated Circuit (ASIC), or the like. The processor may be configured to run and/or execute application processes and/or other modules, processes and/or functions associated with the system and/or a network associated therewith. The underlying device technologies may be provided in a variety of component types including metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, or the like.

The memory may store data as any file suitable for use with the processor including, but not limited to, CSV, HDF, Parquet, ORC, Avro, Pickle, Feather, and the like. In some variations, the memory may include a database (not shown) and may be, for example, an SQLite database, a random-access memory (RAM), a memory buffer, a hard drive, an erasable programmable read-only memory (EPROM), an electrically erasable read-only memory (EEPROM), a read-only memory (ROM), Flash memory, combinations thereof, or the like. As used herein, database refers to a data storage resource. The memory may store instructions to cause the processor to execute modules, processes, and/or functions associated with the system, such as timing of radiation delivery. In some variations, storage may be network-based and accessible for one or more authorized users. Network-based storage may be referred to as remote data storage or cloud data storage. Sensor signal and attachment data stored in cloud data storage (e.g., database) may be accessible to respective users via a network, such as the Internet. In some variations, the database may be a cloud-based FPGA. In some variations, a database may include a medical database (e.g., electronic medical record database). For example, an electronic medical record (EMR) database comprising medical history and information relating to one or more radiotherapy patients may be stored on the memory. Alternatively, one or more patient EMRs may be accessed online through a web browser (e.g., Google Chrome®, Mozilla®, Safari®, Microsoft Edge®, etc.) rendered on a computing device (e.g., computer 312 as shown in FIG. 3). In yet another alternative variation, an EMR may be stored on a third-party database that may be accessed via a computing device.

Some variations described herein relate to a computer storage product with a non-transitory computer-readable medium (i.e., a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for a specific purpose or purposes. Nonlimiting examples of non-transitory computer-readable media include, magnetic storage media such as hard disks, floppy disks, and magnetic tape; optical storage media such as Compact Disc/Digital Video Discs (CD/DVDs); Compact Disc-Read Only Memories (CD-ROMs); holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices.

The systems, devices, and methods described herein may be performed by software (executed on hardware), hardware, or a combination thereof. For example, software may be used to filter patient motion data and control delivery of radiation to a patient during a radiotherapy treatment session, as described in further detail below. Hardware modules may include, for example, a general-purpose processor (or microprocessor or microcontroller), a field programmable gate array (FPGA), an application specific integrated circuit (ASIC), or the like. Software modules (executed on hardware) may be expressed in a variety of software languages (e.g., computer code), including MATLAB, C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The controller may generally include at least one communication module (e.g., communication module 306 as shown in FIG. 3), such as a wireless communication module and/or one or more electrical cables to communicate with one or more devices within the radiotherapy treatment system (e.g., gantry and the patient platform). The system may additionally or alternatively include a communication module that is separate from the controller. For example, the communication module may include a wireless transceiver integrated with the controller configured to receive patient motion data from embedded patient platform sensors and to transmit data to a communication interface integrated with the gantry (e.g., communication interface 105 as shown in FIG. 1). A plurality of signals including radiation beam delivery timing may be transmitted across the communication module simultaneously.

Wireless communication in a wireless network may use any of a plurality of communications standards, protocols and technologies, including but not limited to Bluetooth, near-field communication (NFC), radio-frequency identification (RFID), Wireless Fidelity (Wi-Fi) (e.g., IEEE 802.11a, IEEE 802.11b, IEEE 802.11g, IEEE 802.11n), Wi-MAX, or any other suitable communication protocol. In some variations, a wireless network may connect to a wired network to interface with the Internet, other carrier voice and data networks, business networks, and personal networks. A wired network is typically carried over copper twisted pair, coaxial cable, and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). As used herein, network refers to any combination of wireless, wired, public, and private data networks that are typically interconnected through the Internet, to provide a unified networking and information access system.

As described above, the patient motion detection systems described herein may be integrated with radiotherapy treatment systems to modify (either by a medical professional or automatically) the therapy delivered to a patient. Additionally, or alternatively, a patient motion detection system may be used by a medical professional to augment or further inform their decisions with respect to timing delivery of the radiation to the patient. Accordingly, in some variations, a patient motion detection system may include a display capable of supporting a user interface (e.g., display 308 and user interface 310 as shown in the functional block diagram in FIG. 3) which may allow a medical professional to view, modify, or otherwise interact with radiotherapy treatment data and/or controls. In some variations, the user interface may display recommendations for therapy modifications. The user interface may be displayed on a suitable computing device, which may interface with one or more medical databases (e.g., EMR). Non-limiting examples of a suitable computing device include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iphone®, Samsung Galaxy®, Google Pixel®, etc.), and the like. In some variations, the display may be a touchscreen display configured to respond to a contact force by a user. A touchscreen display may allow for more efficient interaction with the user interface and further simplify a radiotherapy treatment workflow. In some variations, a patient motion detection system may provide sensor status, controller status, and/or other suitable information directly via the user interface (e.g., display, audio feedback, visual feedback, etc.).

### Patient Motion Detection Method

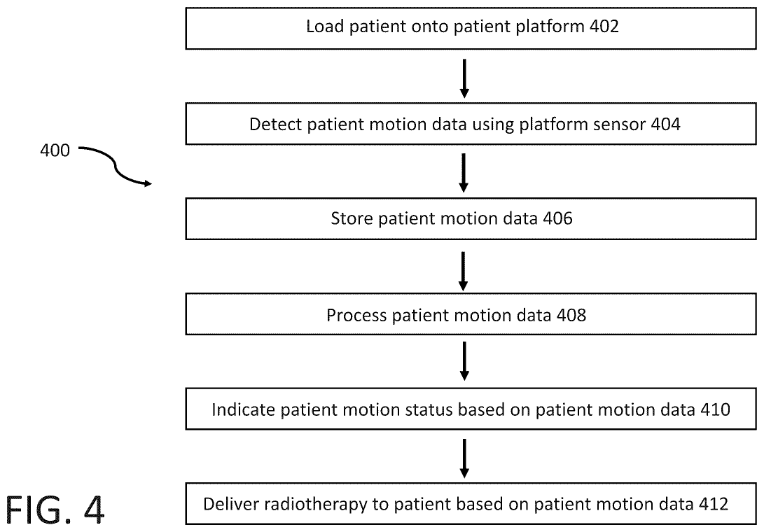

Described below are methods of operation and use of a patient motion detection system that is integrated with a radiotherapy system. In some variations, the methods may be used to help delivery of a radiation dose to a treatment region of a patient. FIG. 4 is a flowchart representation of one variation of a method for measuring patient motion using a radiotherapy patient motion detection system. While FIG. 4 shows that each step of method 400 occurs one time, it should be understood that method 400 may be a continuous process having feedback loops between steps (e.g., a feedback loop may exist between indicate 410 patient motion status and detect 404 patient motion data), may include optional steps (i.e., deliver 412 radiotherapy to patient), and/or may include additional steps.

In one variation, method 400 may comprise loading 402 a patient onto a patient platform. One or more sensors mounted to and/or embedded within the patient platform may then detect 404 patient motion data. A sensor may detect motion data continuously at a predetermined rate. Additionally, or alternatively, the sensor may detect patient motion data discretely. For example, the sensor may be queried at any time by a medical professional to detect motion data. In some variations, the predetermined rate at which data may be captured by the sensor may be adjustable. Next, the detected patient motion data may be stored 406. The sensor may transmit the patient motion data to a controller including a memory configured to store the data. In some variations, the sensor itself may include a memory configured to store the data. Then, the patient motion data may be processed 408. As described in further detail below, processing sensor data may include filtering and/or characterizing the electrical outputs of the sensor to assess changes in patient position caused by patient motion. In some variations, the controller may include one or more processors configured to process the patient motion data. Next, the processed patient motion data may be used to indicate 410 a motion status of the patient. For example, a signal indicative of the processed motion data may be transmitted to a user interface on a display to represent the patient's motion. A representation of patient motion may be graphical and may typically be interpretable to a medical professional. In some variations, the processed patient motion data may be used to generate a notification of unsafe and/or safe levels of patient motion. The notification may inform a medical professional about patient motion via digital, visual, aural, or haptic outputs (or combinations thereof), and may be produced via the controller or a device communicably coupled to the controller (e.g., a computer). A medical professional, or an integrated radiotherapy treatment system, may decide whether to modify radiotherapy delivered to the patient based on the indication of patient motion status. In some variations, a notification indicative of unsafe levels of patient motion may differ from a notification indicative of safe levels of patient motion. For example, a notification of a large patient motion may be represented by the activation of a red LED on the controller, while a notification of no patient motion may be represented by activation of a green LED on the controller. The red LED may indicate to a medical professional that it is unsafe to deliver radiotherapy, while the green LED may indicate that it is safe to continue the radiotherapy treatment. Typically, unsafe levels of patient motion may correspond to motion data that is above a predetermined threshold or outside of a predetermined range of acceptable values. Finally, a radiation beam to treat a region on the patient may optionally be modified 412 depending on the processed patient motion data and indicated motion status of the patient. Additional aspects of radiotherapy treatment modification based on patient motion detection are described in further detail below.

Modifying radiotherapy based on patient motion data may comprise processing patient motion data and using the processed patient motion data to automatically and/or intentionally adjust or delay delivery of a radiation beam to a treatment region on a radiotherapy patient. Automatic adjustments to radiotherapy may be accomplished via a controller which may analyze the processed motion data to determine changes in position of the treatment region due to patient motion (e.g., using a software program that is configured to execute any of the methods described herein) and subsequently transmit a control signal to a radiation source and/or communication interface within the radiotherapy treatment system (e.g., radiation source 110, communication interface 105 in FIG. 1). Intentional adjustments to radiotherapy may be accomplished by a medical professional via a user interface, which may display the processed patient motion data and allow the medical professional to choose whether to send a control signal and/or what information the control signal may transmit to the treatment system. For example, the control signal may include a command to stop delivery of an existing radiation treatment beam, delay delivery of a radiation treatment beam for an amount of time (e.g., seconds, minutes), initiate delivery of a radiation treatment beam, or change a property of the beam (e.g., pattern, duration, energy, etc.). In some variations, a control signal may comprise a patient platform control signal that moves the patient platform in a way that compensates for the patient's position (e.g., change in center of mass). For example, if the patient's center of mass moves in one direction along the y-axis by a certain distance, the control signal may move the patient platform in the opposite direction on the y-axis by the same distance. The control signal may be used to delay delivery of a radiation treatment beam until, or initiate a radiation treatment beam during, a breath-hold of a patient. This strategy may minimize radiation delivered to healthy tissue, which may have moved into a patient's target treatment zone during the patient motion. In some variations, the control signal may be used to delay or stop delivery of a radiation beam while a patient is moving (e.g., coughing, sitting up, moving a body part, etc.).

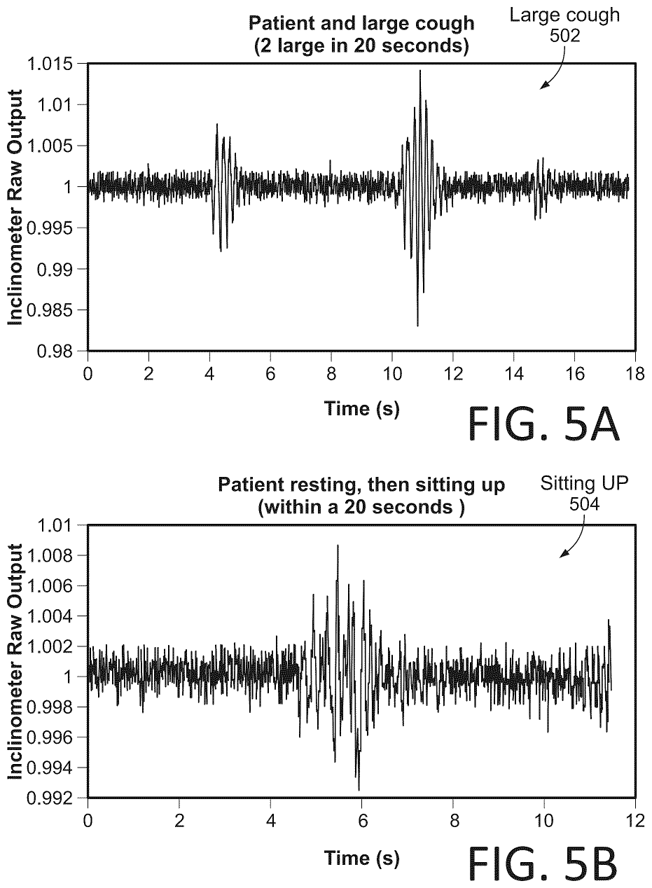

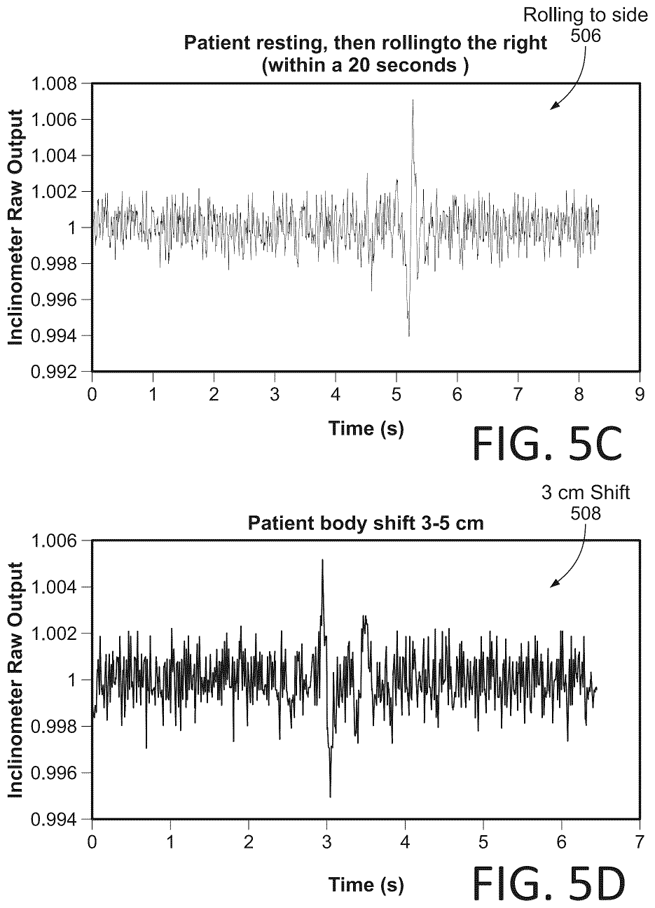

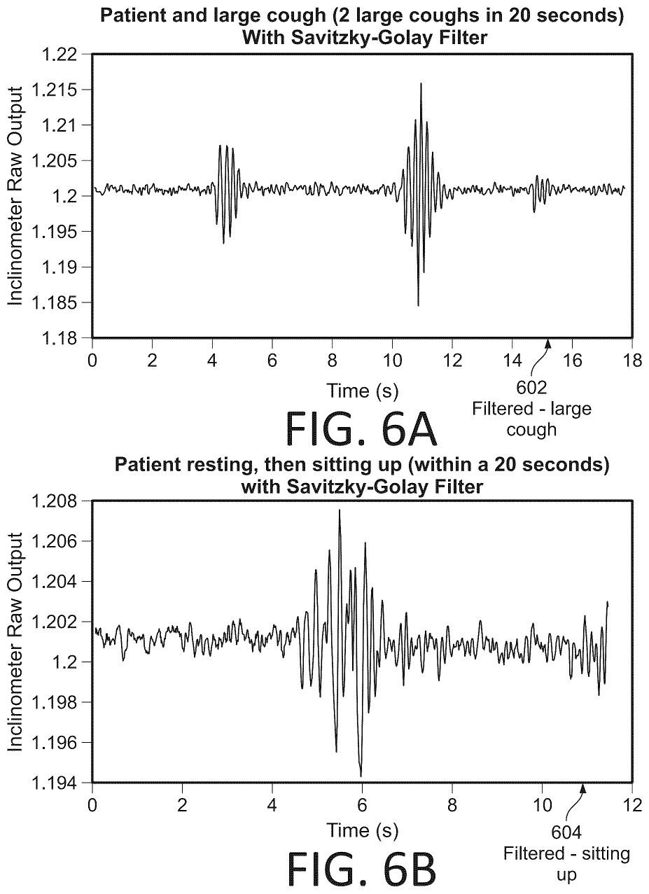

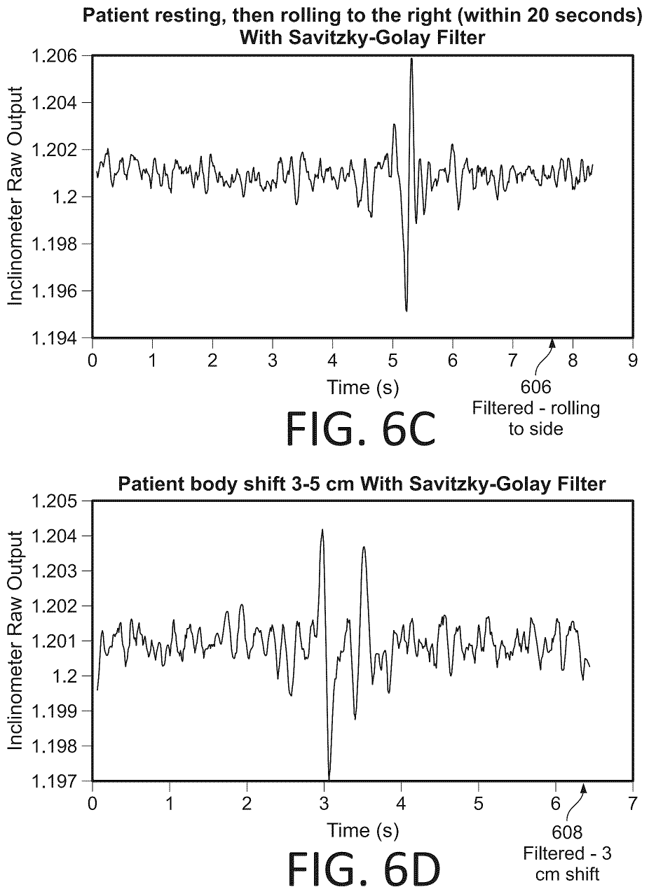

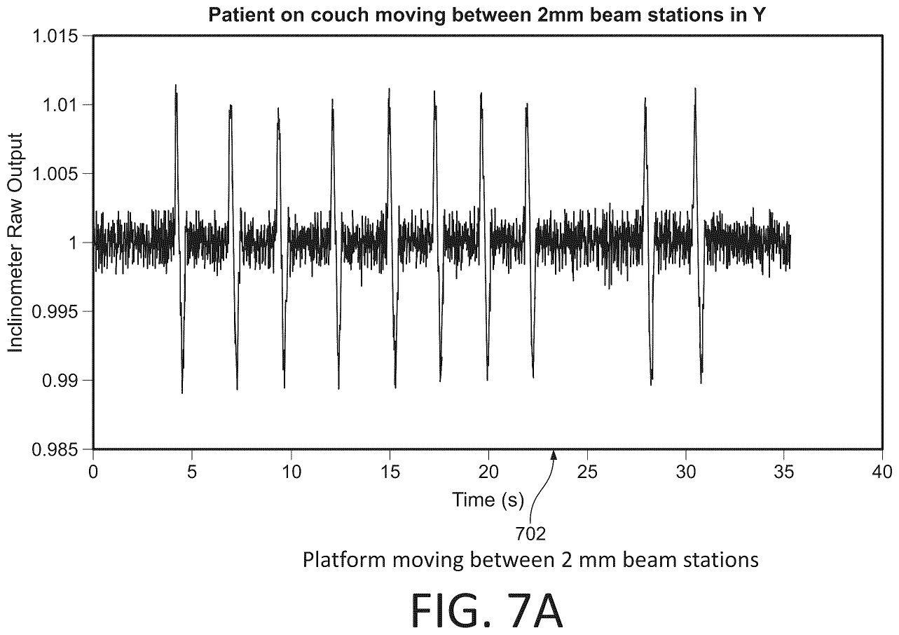

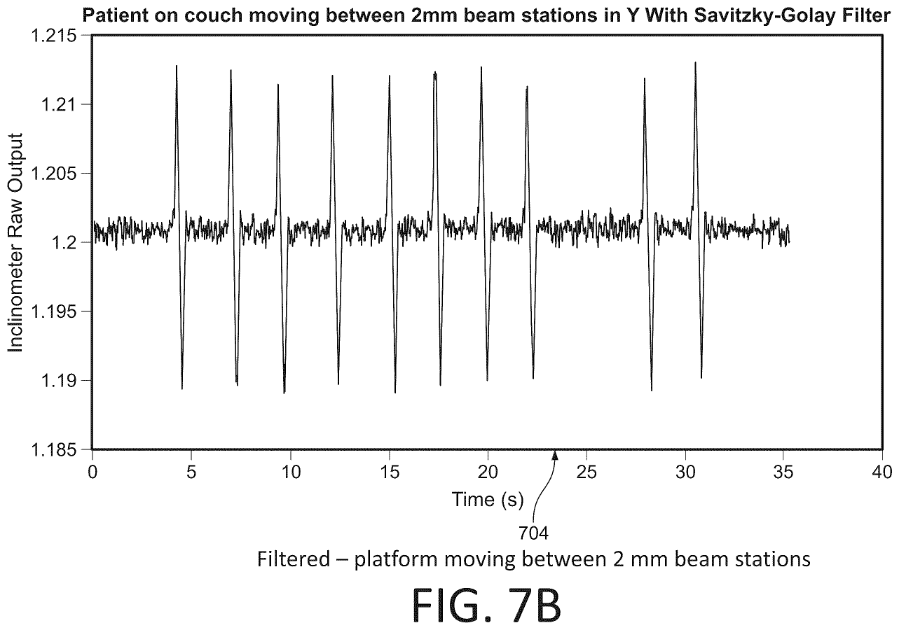

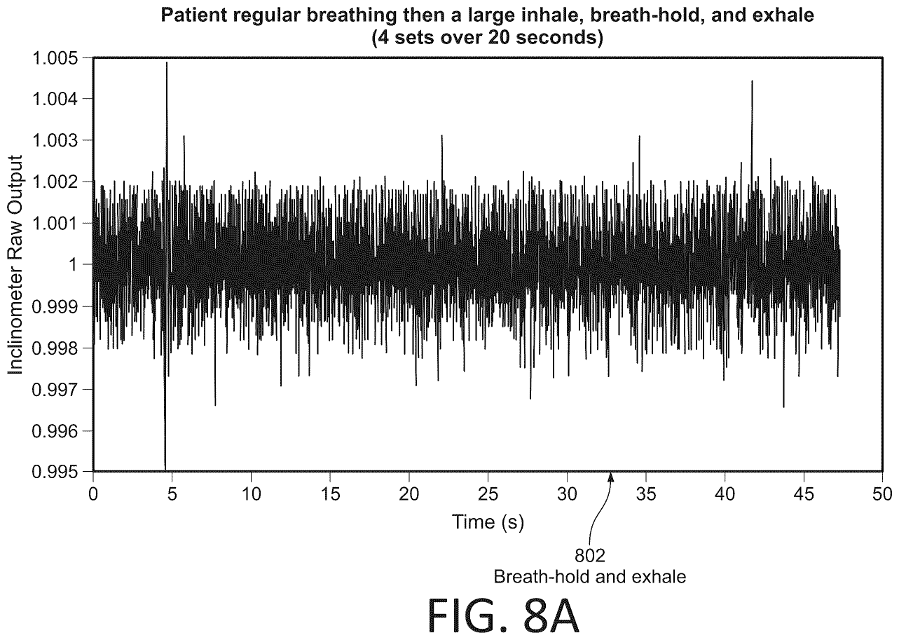

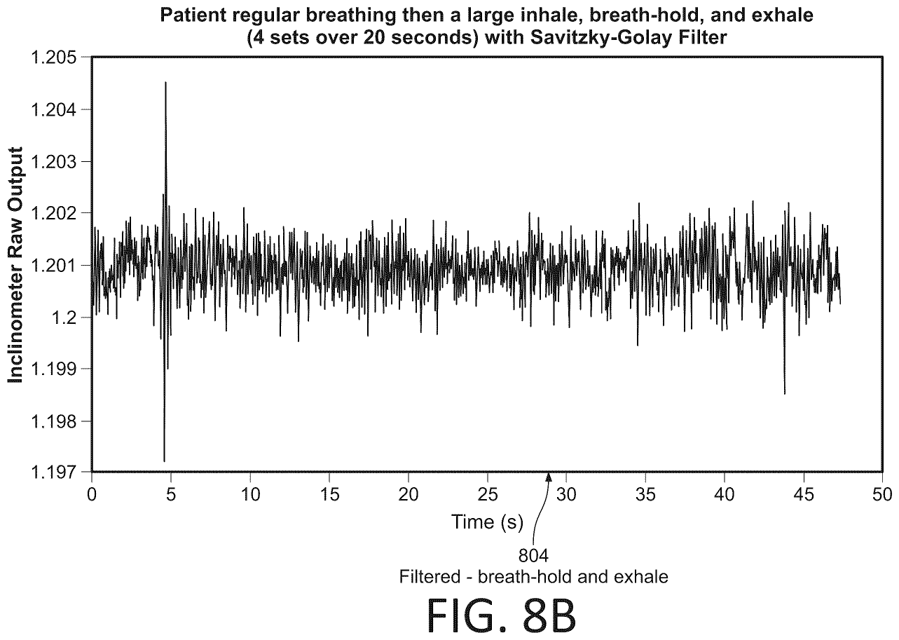

Processing the patient motion data (e.g., step 408 of method 400) may include graphically visualizing the data and applying a filter or other processing function to the data to increase the signal-to-noise ratio of the data and more accurately determine whether the data indicates or represents patient motion. Graphs may be rendered, and filters may be applied using any suitable software, such as MATLAB. For example, FIG. 5 shows patient motion data (large cough 502, sitting up 504, rolling to side 506, and 3 cm shift 508) plotted on graphs of sensor output (i.e., a current trace from an inclinometer) versus time. FIG. 5A depicts the inclinometer sensor output (502) during an experiment where a patient had two large coughs in 20 seconds. FIG. 5B depicts the inclinometer sensor output (504) acquired during an experiment where a patient was resting and then sat up within 20 seconds. FIG. 5C depicts the inclinometer sensor output (506) acquired during an experiment where a patient was resting and then rolled to the right within 20 seconds. FIG. 5D depicts the inclinometer sensor output (508) acquired during an experiment where a patient shifted their body about 3-5 cm. FIGS. 6A-6D show corresponding plots of filtered patient motion data (large cough 602 in FIG. 6A, sitting up 604 in FIG. 6B, rolling to side 606 in FIG. 6C, and 3 cm shift 608 in FIG. 6D), which are notably less noisy. In this example and the examples in FIGS. 7-8, the sensor output from the inclinometer was filtered using a Savitzky-Golay filter. Similarly, FIG. 7A shows an unfiltered (702) plot of inclinometer sensor output for patient motion occurring between delivery of radiation beams (i.e., the patient was on a couch that was moving between beam stations along the Y-axis in 2 mm intervals), and FIG. 7B depicts a plot (704) of the filtered inclinometer sensor output, where the filtered plot (704) contains less noise. FIG. 8A shows an unfiltered plot (802) of inclinometer sensor output of a patient breath-hold and exhale (i.e., the patient was breathing regularly and then did a large inhale, breath-hold, and exhale, repeatedly 4 times over 20 seconds), and FIG. 8B depicts a plot (804) of the filtered inclinometer sensor output, where the filtered plot (804) includes less noise. Thus, the patient motion may be easier to identify by a medical professional and/or computer program. The filtered plots shown in FIGS. 6-8 may be generated by applying first-order filters (and/or any of the filters disclosed herein) to sensor (e.g., inclinometer) data to reduce noise levels and/or amplify or highlight data that may correspond to patient motions.

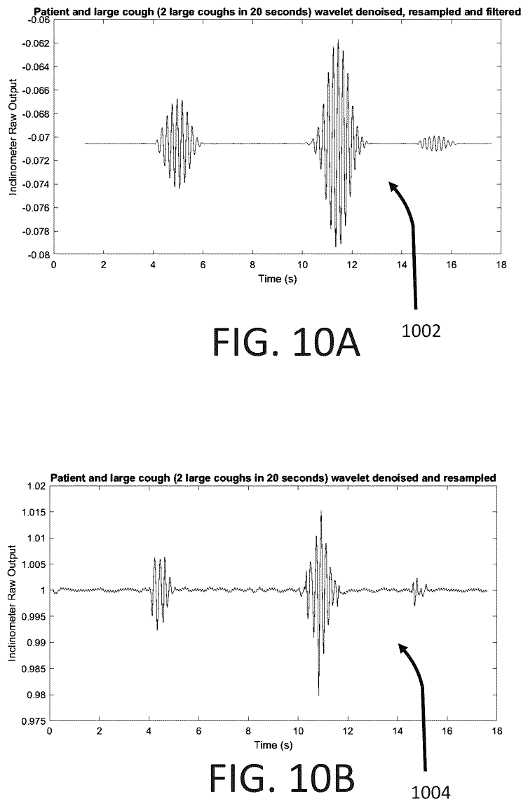

Alternate and/or additional methods of filtering and processing may be applied to the acquired sensor data to extract patient motion data. FIG. 10A depicts patient motion data (1002) that was generated from inclinometer sensor output that was wavelet denoised, resampled, and filtered, where the inclinometer sensor output was acquired during an experiment where a patient had two large coughs in 20 seconds. Wavelet denoising of a signal may comprise decomposing the signal into different frequency bands using a wavelet transform, then the wavelet coefficients that represent noise are suppressed, leaving the signals corresponding to the non-noise frequency bands. After the sensor output has been denoised, it may be further processed using a bandpass filter, such as a FIR (finite impulse response) window bandpass filter. FIG. 10B depicts patient motion data (1004) that was generated from the same inclinometer sensor output as in FIG. 10A, but the signal was wavelet denoised and resampled (without filtering). Resampling of the sensor signal may help remove certain noise artifacts.

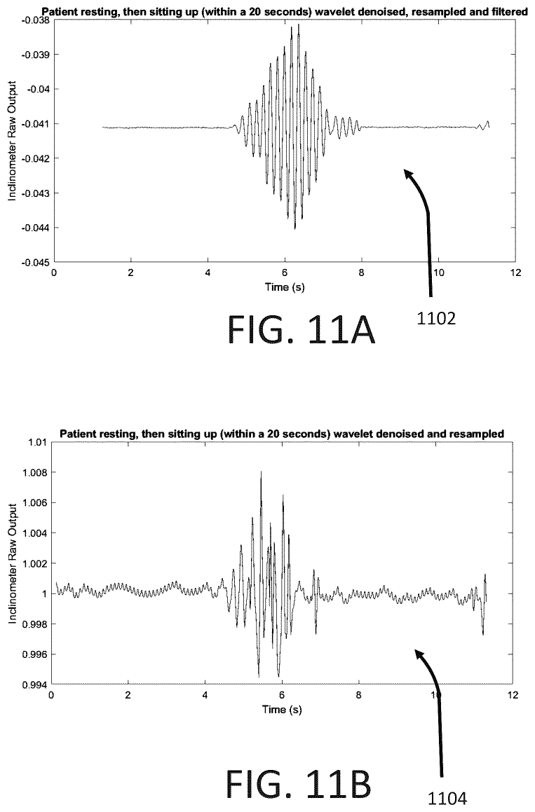

FIG. 11A depicts patient motion data (1102) that was generated from inclinometer sensor output that was wavelet denoised, resampled, and filtered (as in FIG. 10A), where the inclinometer sensor output was acquired during an experiment where a patient was resting and then sits up within 20 seconds. FIG. 11B depicts patient motion data (1104) that was generated from the same inclinometer sensor output as in FIG. 11A, but the signal was wavelet denoised and resampled (without filtering, as in FIG. 10B).

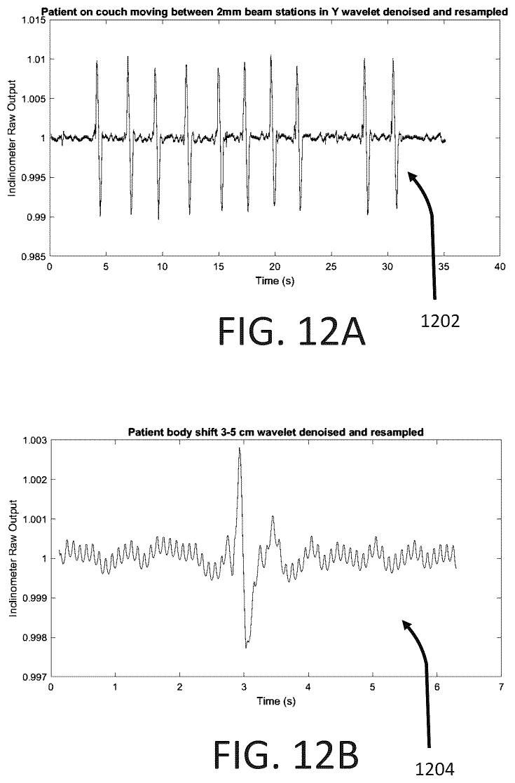

FIG. 12A depicts patient motion data (1202) that was generated from inclinometer sensor output that was wavelet denoised and resampled, where the inclinometer sensor output was acquired during an experiment where a patient was on a couch that was moving between beam stations along the Y-axis in 2 mm intervals. FIG. 12B depicts patient motion data (1204) that was generated from inclinometer sensor output that was wavelet denoised and resampled, where the inclinometer sensor output was acquired during an experiment where a patient shifted their body about 3-5 cm.

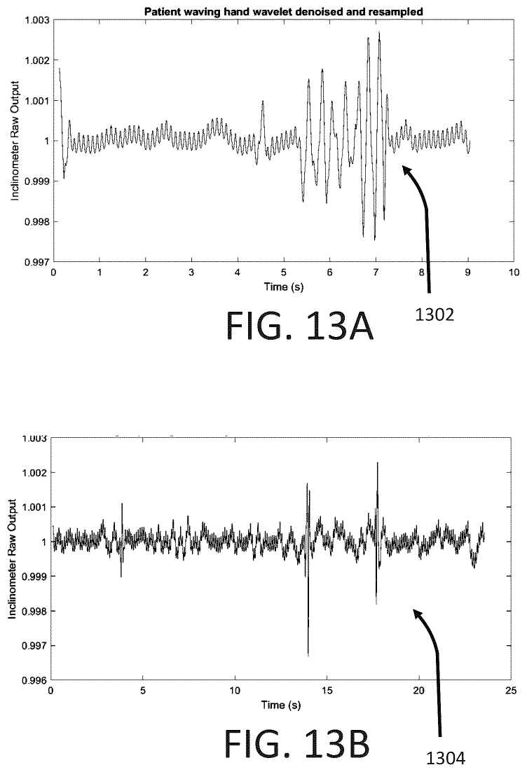

FIG. 13A depicts a further example of a plot of patient motion data (1302) that was generated from inclinometer sensor output that was wavelet denoised and resampled, where the inclinometer sensor output was acquired during an experiment where a patient was on a couch and waved their hand. FIG. 13B depicts patient motion data (1304) that was generated from inclinometer sensor output that was wavelet denoised and resampled, where the inclinometer sensor output was acquired during an experiment where a patient had multiple small coughs every few seconds for a total of 20 seconds.

Any higher order and lower order filters (e.g., the first-order filters of the examples described herein) alone or in combination, may be applied to acquired sensor data from the one or more sensors described herein to improve the quality of the patient motion data acquired, including low-pass, high-pass, band-pass, and notch filters. In some variations, filters such as adaptive filters and/or Fourier transforms and/or wavelet denoising may additionally or alternatively be applied to process the motion data. For example, applying a Fourier transform to the sensor signal may help extract signals having frequencies that correspond with the frequencies of certain types of patient motion. The sensor (e.g., inclinometer) signal may be decomposed into its different frequency components and the methods described herein may comprise extracting the frequency components that correspond to breathing motion (e.g., at a rate of 12-20 breaths per minute), and/or extracting the frequency components that correspond to peristalsis (e.g., at a rate of 3-12 contractions per minute), and/or extracting frequency components that correspond to sudden shifts (e.g., due to a twist, cough, sneeze). Alternatively, or additionally, some variations may include the use of one or more convolutional neural networks (CNN) and/or deep learning methods and/or machine learning methods in conjunction with patient “training data” to process platform sensor data in order to detect and/or extract patient motion data. Examples of neural networks that may be used with any of the methods described herein may include, but are not limited to, recurrent neural networks (RNN) including long short-term memory networks (LSTM), generative adversarial networks (GAN), and/or multilayer perceptrons (MLP). The processed platform sensor data and/or processed patient motion data may then be used to determine any actions (such as beam holds etc.) to be taken during treatment. In some variations, such as variations in which a controller automatically modifies a radiotherapy based on patient motion data, the raw patient motion data may not require visualization.

In some variations, processing the patient motion data may comprise determining whether the data is under a predetermined threshold or within a predetermined range of acceptable values. In such variations, unsafe patient motion may correspond to the identification of patient motion data over the predetermined threshold or outside of the predetermined range of acceptable values.

Processing the data may optionally include characterizing the type of patient motion exhibited by the data. For example, patient motion may be characterized as large-scale or small-scale. Large scale movements may include moving one or more body part, coughing, sneezing, rolling over, sitting up, and the like. Small-scale movements may include normal respiration cycles, breath-holds, and the like. Characterizing the patient motion as large-scale or small-scale may simplify modification of the radiotherapy delivered to a patient. For example, the ability to distinguish between a patient breathing motion and a generally large-scale patient motion may simplify a decision process for if and/or how to modify the radiotherapy.

Additionally, or alternatively, processing patient motion data may include transforming the data into a different domain and analyzing the data in the new domain to identify and/or characterize patient motion. For example, raw patient motion data collected by a sensor in a patient platform may be transmitted to a controller, stored in a memory, and transformed into the frequency domain via a processor. Then, the transformed frequency motion data may be assessed to identify any frequency ranges corresponding to a type of patient motion (e.g., 12-20 changes/minute may indicate respiration). In some variations, processing patient motion data may comprise processing the acquired sensor data using short-time Fourier transform. For example, the acquired sensor data may be cut or divided into shorter data segments that may be transformed into the frequency domain and/or processed as described above. Segmenting the sensor data may facilitate more frequent sampling and faster sensing of whether a patient moved or not. For example, inclinometer sensor data acquired over 20 seconds may be divided into 40 data segments of 0.5 second per segment, which may provide information about patient motion more rapidly so that the system and/or clinician may respond to the patient motion in real-time (or near real-time). In some variations, the transformed patient motion data may be graphically displayed.

### ENUMERATED EMBODIMENTS

Embodiment I-1. A system for detecting patient motion during a radiotherapy treatment session, comprising:
a gantry comprising a radiation source,
a patient platform comprising a patient support surface and configured to translate laterally through the gantry, and
a sensor coupled to the patient support surface, wherein the patient platform sensor is configured to collect motion data of a patient on the patient support surface.

Embodiment I-2. The system of Embodiment I-1, wherein the sensor is communicably coupled to a controller comprising a memory configured to store the motion data.

Embodiment I-3. The system of Embodiment I-1, wherein the sensor comprises a memory configured to store the motion data.

Embodiment I-4. The system of Embodiment I-2, wherein the motion data is stored in the memory as a CSV file.

Embodiment I-5. The system of Embodiment I-1, wherein the motion data comprises a magnitude of one or more of a slope, an elevation, a depression, a force, or a strain of the patient platform.

Embodiment I-6. The system of Embodiment I-1, wherein the motion data is used to modify a radiation dose delivered to the patient.

Embodiment I-7. The system of Embodiment I-1, wherein the patient platform sensor comprises a motion sensor.

Embodiment I-8. The system of Embodiment I-1, wherein the patient platform sensor comprises an inclinometer.

Embodiment I-9. The system of Embodiment I-8, wherein the inclinometer is configured to measure an angle of one or more of a slope, an elevation, or a depression of the patient platform.

Embodiment I-10. The system of Embodiment I-8, wherein a resolution of the inclinometer is between about 1 μradian and about 5 μradians.

Embodiment I-11. The system of Embodiment I-1, wherein the patient platform sensor collects the motion data at a rate of between about 1 Hz and about 100 Hz.

Embodiment I-12. The system of Embodiment I-1, wherein the patient platform sensor collects the motion data at a rate of at least 50 Hz.

Embodiment I-13. A radiotherapy system for detecting patient motion comprising:
a platform configured to support a patient during a radiotherapy treatment session, the patient platform comprising a patient support surface and a sensor coupled to the patient support surface, wherein the sensor is configured to collect data on the patient motion during the radiotherapy treatment session.

Embodiment I-14. The system of Embodiment I-13, wherein the sensor is communicably coupled to a controller comprising a memory configured to store the data.

Embodiment I-15. The system of Embodiment I-13, further comprising a processor configured to apply a filter to the data.

Embodiment I-16. The system of Embodiment I-15, wherein the filter comprises a first-order filter.

Embodiment I-17. The system of Embodiment I-13, wherein the data is used to modify a radiation dose delivered to the patient.

Embodiment I-18. The system of Embodiment I-13, wherein the sensor comprises an inclinometer.

Embodiment I-19. The system of Embodiment I-18, wherein the inclinometer is configured to measure an angle of one or more of a slope, an elevation, or a depression of the patient platform.

Embodiment I-20. The system of Embodiment I-18, wherein a resolution of the inclinometer is about 3.5 μradians.

Embodiment I-21. A method for detecting patient motion during a radiotherapy session, comprising:
collecting patient motion data from a patient on a patient platform, wherein the patient platform comprises a patient support surface and a sensor coupled thereto, and wherein the patient motion data is collected by the sensor,
storing the patient motion data, and
optionally modifying a radiation therapy delivered to the patient based at least in part on the patient motion data.

Embodiment I-22. The method of Embodiment I-21, wherein modifying the radiation therapy comprises delivering a radiotherapy beam to the patient during a specific breathing phase of the patient.

Embodiment I-23. The method of Embodiment I-22, wherein a specific breathing phase of the patient comprises a breath-hold.

Embodiment I-24. The method of Embodiment I-21, wherein modifying the radiation therapy comprises respiratory gating.

Embodiment I-25. The method of Embodiment I-21, wherein modifying the radiation therapy comprises delaying a delivery of a radiotherapy beam during a large motion made by the patient.

Embodiment I-26. The method of Embodiment I-21, wherein the sensor is configured to detect the data on the patient motion at a rate of about 80 Hz.

Embodiment I-27. The method of Embodiment I-21, wherein the patient motion comprises one or more of a breath, a cough, a torso adjustment, a limb adjustment, and a head adjustment.

Embodiment I-28. The method of Embodiment I-21, wherein the patient motion comprises a change in a center of mass of the patient with respect to the patient platform.

Embodiment I-29. The method of Embodiment I-28, wherein the change in the center of mass of the patient causes a change in a strain field of the patient platform.

The foregoing description, for purposes of explanation, used specific nomenclature to provide a thorough understanding of the invention. However, one skilled in the art will understand that specific details are not required to practice the invention. Thus, the foregoing descriptions of specific embodiments of the invention are presented for purposes of illustration and description, and not intended to be exhaustive or to limit the invention to the precise forms disclosed. Indeed, many modifications and variations are possible in view of the above teachings. The embodiments were chosen and described to explain the principles of the invention and its practical applications and thereby enable others skilled in the art to utilize the invention and various embodiments with various modifications as suited to the particular use contemplated. It is intended that the following claims and their equivalents define the scope of the invention.

1. The system of claim 13, further comprising
a gantry comprising a radiation source.

2. The system of claim 13, wherein the sensor is communicably coupled to a controller comprising a memory configured to store the motion data.

3. (canceled)

4. (canceled)

5. The system of claim 13, wherein the motion data comprises a magnitude of one or more of a slope, an elevation, a depression, a force, or a strain of the patient platform.

6. The system of claim 13, wherein the motion data is used to modify a radiation dose delivered to the patient.

7. The system of claim 13, wherein the sensor comprises a motion sensor.

8. The system of claim 13, wherein the sensor comprises an inclinometer.

9. The system of claim 8, wherein the inclinometer is configured to measure an angle of one or more of a slope, an elevation, or a depression of the patient platform.

10. The system of claim 8, wherein a resolution of the inclinometer is between about 1 μradian and about 5 μradians.

11. The system of claim 13, wherein the sensor collects the motion data at a rate of between about 1 Hz and about 100 Hz.

12. The system of claim 13, wherein the platform-sensor collects the motion data at a rate of at least 50 Hz.

13. A radiotherapy system for detecting patient motion comprising:
a platform configured to support a patient during a radiotherapy treatment session, the patient platform comprising a patient support surface and a sensor coupled to the patient support surface, wherein the sensor is configured to collect motion data of the patient during the radiotherapy treatment session.

14. The system of claim 13, wherein the sensor is communicably coupled to a controller comprising a memory configured to store the data.

15. The system of claim 13, wherein the controller further comprises a processor configured to apply a filter to the data.

16. The system of claim 15, wherein the filter comprises a first-order filter.

17. The system of claim 13, wherein the data is used to modify a radiation dose delivered to the patient.

18. (canceled)

19. (canceled)

20. The system of claim 8, wherein a resolution of the inclinometer is about 3.5 μradians.

21. The system of claim 13, wherein the controller further comprises a processor configured to wavelet denoise and resample the data.

22. A method for detecting patient motion during a radiotherapy session, comprising:
collecting patient motion data from a patient on a patient platform, wherein the patient platform comprises a patient support surface and a sensor coupled thereto, and wherein the patient motion data is collected by the sensor,
storing the patient motion data, and
optionally modifying a radiation therapy delivered to the patient based at least in part on the patient motion data.

23. The method of claim 22, wherein modifying the radiation therapy comprises delivering a radiotherapy beam to the patient during a specific breathing phase of the patient.

24. The method of claim 23, wherein a specific breathing phase of the patient comprises a breath-hold.

25. The method of claim 22, wherein modifying the radiation therapy comprises respiratory gating.

26. The system of claim 22, wherein modifying the radiation therapy comprises delaying a delivery of a radiotherapy beam during a large motion made by the patient.

27. The method of claim 22, wherein the sensor is configured to detect the data on the patient motion at a rate of about 80 Hz.

28. The system of claim 22, wherein the patient motion comprises one or more of a breath, a cough, a torso adjustment, a limb adjustment, and a head adjustment.

29. The system of claim 22, wherein the patient motion comprises a change in a center of mass of the patient with respect to the patient platform.

30. The system of claim 29, wherein the change in the center of mass of the patient causes a change in a strain field of the patient platform.
